(19) United States
(12) Patent Application Publication
(10) Pub. No.: US 2021/0041408 A1
(43) Pub. Date: Feb. 11, 2021

(54) SWITCHGEAR CABINET FOR EXHAUST GAS MEASURING FACILITIES

(71) Applicant: AVL EMISSION TEST SYSTEMS GMBH, NEUSS (DE)

(72) Inventor: ACHIM DICKOW, VELBERT (DE)

(73) Assignee: AVL EMISSION TEST SYSTEMS GMBH, NEUSS (DE)

(21) Appl. No.: 16/069,519

(22) PCT Filed: Dec. 9, 2016
(86) PCT No.: PCT/EP2016/080417

§ 371 Date: Jul. 12, 2018

(30) Foreign Application Priority Data
Jan. 15, 2016 (DE) 10 2016 100 634.3

(51) Int. Cl. G01N 33/00; G01N 27/62; G01N 21/76

(52) U.S. Cl. CPC G01N 33/0032; G01N 21/766; G01N 27/626

(57) ABSTRACT

A control cabinet for exhaust gas measuring facilities. The control cabinet includes a first cabinet body with first measuring appliances arranged therein for analyzing at least one sample gas, a door which opens and closes an open front side of the first cabinet body, the door being rotatably attached to the first cabinet body, an operating unit arranged on the door, and a second cabinet body with second measuring appliances arranged therein. The second cabinet body includes a second front side that can be closed and opened via a guided relative movement of the first cabinet body in relation to the second cabinet body. The first cabinet body is movable in a guided manner relative to the second cabinet body.

### CROSS REFERENCE TO PRIOR APPLICATIONS

This application is a U.S. National Phase application under 35 U.S.C. § 371 of International Application No. PCT/EP2016/080417, filed on Dec. 9, 2016 and which claims benefit to German Patent Application No. 10 2016 100 634.3, filed on Jan. 15, 2016. The International Application was published in German on Jul. 20, 2017 as WO 2017/121553 A1 under PCT Article 21(2).

### FIELD

The present invention relates to a control cabinet for exhaust gas measuring facilities comprising a first cabinet body in which first measuring appliances for the analysis of sample gases are arranged, a door for opening and closing an open front side of the first cabinet body which is rotatably attached to the first cabinet body, and an operating unit arranged at the door.

### BACKGROUND

Such control cabinets are used, for example, at roller test benches of motor vehicles. These control cabinets comprise the driving and electronic systems of the regulating members in the exhaust gas measuring facility which may be configured, for example, as a CVS facility (constant volume sample), and the measuring appliances required for the analysis of the exhaust gas for determining the pollutant quantities in the exhaust gas, such as flame ionization detector analysis appliances for determining hydrocarbons, or via a gas chromatograph for determining methane quantities, chemiluminescence detector analysis appliances for determining nitrogen oxide quantities in the exhaust gas, infrared detector analysis appliances for determining various active components, such as carbon monoxide, carbon dioxide or hydrocarbon compounds in the exhaust gas. The control cabinet is accordingly connected to the sampling probes of the exhaust gas measuring facility via leads which are connected to the control cabinet via corresponding couplings.

Each control cabinet accordingly comprises the measuring appliances and controls for a measuring line and includes an opening at its door arranged at the front side in which opening the operating unit may be located and via which the various measuring appliances can be operated so that the analysis data are immediately available.

In most facilities for exhaust gas analysis, a plurality of roller test benches, engine test benches and/or exhaust gas measuring lines are, however, accommodated where, in order to save time, measurements on various vehicles or engines are simultaneously carried out. The operating personnel must either change between the control cabinets or operating personnel for each individual facility must be available. The stricter exhaust emission standards also require new measurements so that additional space for accommodating the required appliances is necessary; space problems therefore arise in the halls provided for this purpose.

### SUMMARY

An aspect of the present invention is to provide a control cabinet for exhaust gas measuring facilities via which at least two exhaust gas lines can be simultaneously operated via an operating unit, or where additional measuring appliances can be placed in the cabinet while still being easy to reach. A further aspect of the present invention is that the installation space used should occupy as little space as possible despite a large number of measuring appliances to be used, while all instruments and connections remain easily accessible. A further aspect of the present invention is that the costs for such a control cabinet are reduced compared with previously-described solutions.

In an embodiment, the present invention provides a control cabinet for exhaust gas measuring facilities. The control cabinet includes a first cabinet body configured to have first measuring appliances arranged therein for analyzing at least one sample gas, a door configured to open and close an open front side of the first cabinet body, the door being rotatably attached to the first cabinet body, an operating unit arranged on the door, and a second cabinet body configured to have second measuring appliances arranged therein. The second cabinet body comprises a second front side that can be closed and opened via a guided relative movement of the first cabinet body in relation to the second cabinet body. The first cabinet body is movable in a guided manner relative to the second cabinet body.

### BRIEF DESCRIPTION OF THE DRAWINGS

The present invention is described in greater detail below on the basis of embodiments and of the drawings in which:

FIG. 1 shows a front view of a switchgear for an exhaust gas measuring facility according to the present invention in the closed state of a first cabinet body;

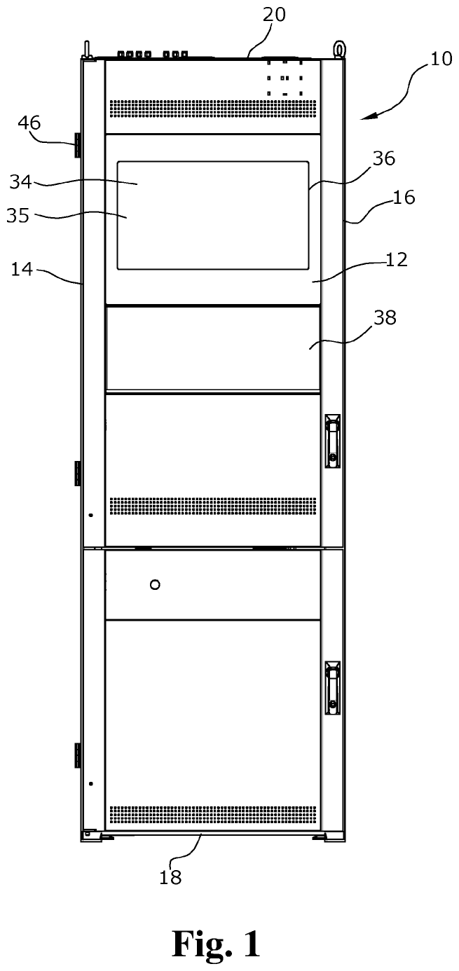

FIG. 2 shows a perspective view of the control cabinet according to the present invention shown in FIG. 1 in the open state of the first cabinet body;

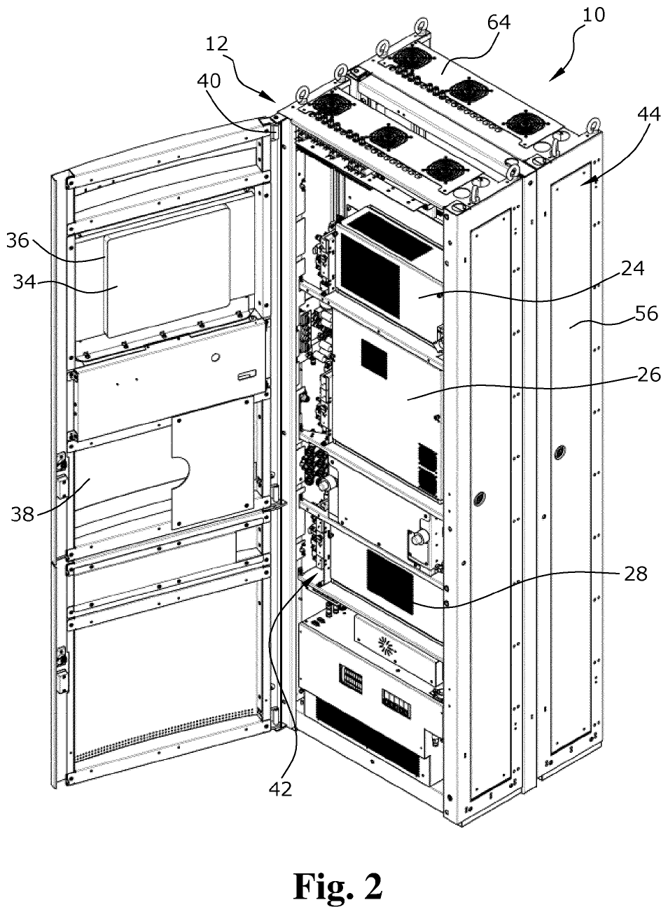

FIG. 3 shows a side view of the control cabinet according to the present invention in the closed state; and

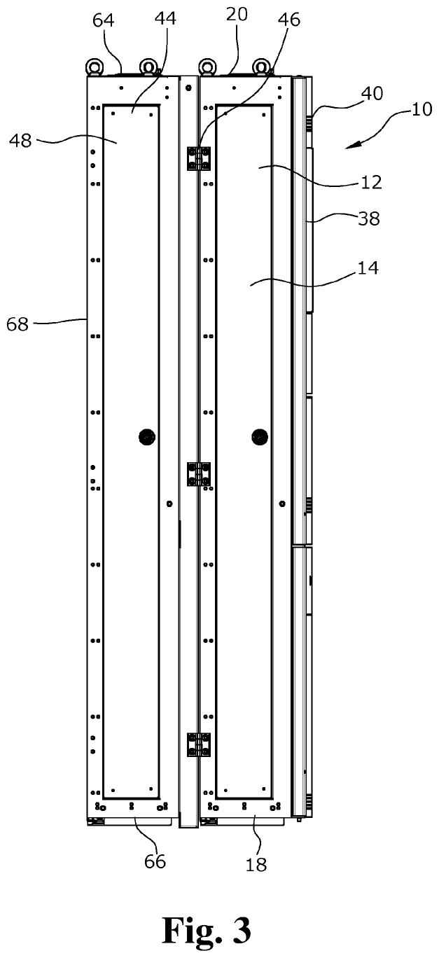

FIG. 4 shows a perspective side view of the control cabinet according to the present invention in the open state of a second cabinet body.

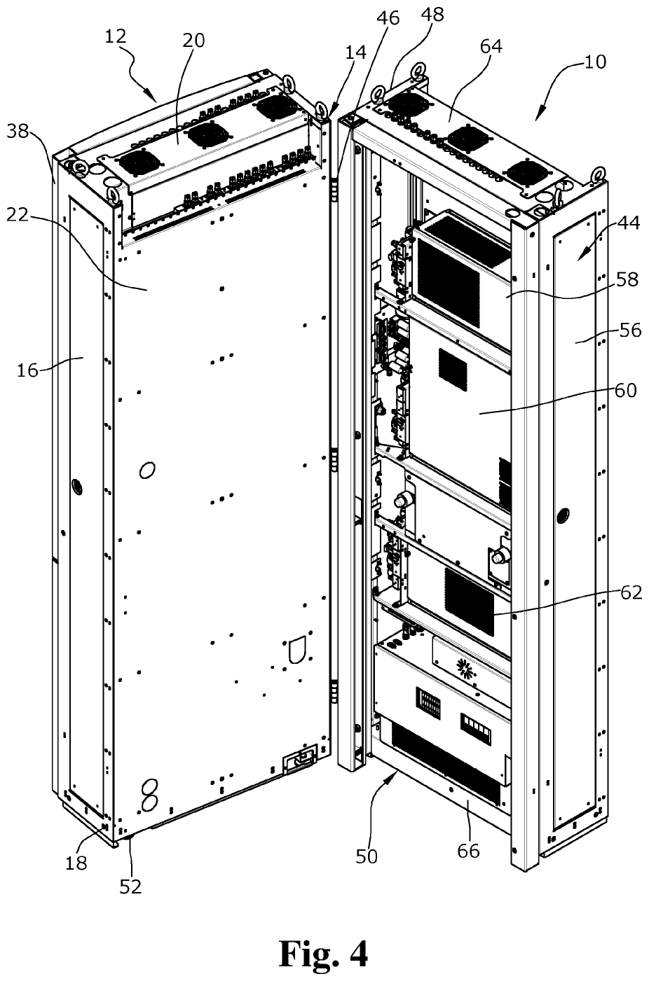

### DETAILED DESCRIPTION

Due to the fact that the first cabinet body is adapted to be moved in a guided manner relative to a second cabinet body in which second measuring appliances are arranged and which includes a second open front side that is adapted to be opened and closed through the guided relative movement of the first cabinet body in relation to the second cabinet body, a compact and space-saving unit is created where good accessibility of both cabinets is provided. The compact unit allows for driving a plurality of measuring lines via a single operating unit. Costs are reduced since a cabinet wall on the front side can be omitted.

In an embodiment of the present invention, the second cabinet body can, for example, be attached to the first cabinet body via an articulated connection, and the first cabinet body can, for example, be arranged relative to the second cabinet body so that it is rotatable about the articulated connection. The control cabinets can accordingly be swung away from each other so that all parts arranged inside the two cabinets are easy to reach.

In an embodiment of the present invention, the measuring appliances in the second cabinet body can, for example, be electrically connected to the operating unit at the door of the first cabinet body. The costs are thus considerably reduced since an operating unit for the second control cabinet can be omitted. Simultaneous operation of two exhaust gas measuring lines is additionally allowed for via a single operating unit which is still easy to access.

In an embodiment of the present invention, the measuring appliances in the first cabinet body can, for example, be assigned to a first exhaust gas line and the measuring appliances in the second cabinet body can, for example, be assigned to a second exhaust gas measuring facility. Two identical facilities can thus be simultaneously operated and controlled via a double control cabinet. An assigning to the corresponding measuring appliances as well as a correct assembly is facilitated if errors occur.

In an embodiment of the present invention, rollers can, for example, be arranged at a bottom of the first cabinet body. These rollers allow for the cabinet body to be swung away from the second cabinet body without exerting much force, thereby improving the accessibility of the second cabinet body.

It is advantageous when the rear wall of the first cabinet body rests against the open front side of the second cabinet body. Two identical cabinet bodies can be used in such an arrangement, whereby the costs can again be reduced because a higher number of identical parts can be used.

In an embodiment of the present invention, the first cabinet body and the second cabinet body as well as the measuring appliances arranged in the first cabinet body and in the second cabinet body are the same. The number of identical parts used is thus maximized so that costs are again reduced and assembly facilitated.

A particularly simple and guided swinging-open process for improved opening of the cabinet, in particular by 180°, for improved accessibility is realized when the first cabinet body is rotatably attached to a first side wall of the second cabinet body via a plurality of hinges arranged at a first side wall, and closing devices are arranged at the respective opposite second side walls of the two cabinet bodies, via which the second side wall of the first cabinet body is adapted to be connected to the second side wall of the second cabinet body. The second cabinet body is thus allowed to be completely opened in a guided manner.

In an embodiment of the present invention, the operating unit can, for example, include a monitor, in particular a touchscreen, which is arranged in an opening of the door. This allows for a simple guided operation and offers a clearer representation of the produced measured data.

A control cabinet for exhaust gas measuring facilities is thus provided where a plurality of exhaust gas measuring lines can be operated in parallel and simultaneously, the control of which can be carried out by one operator through an operating unit merely via the one control cabinet. The installation space used as well as the costs for setup and assembly are considerably reduced compared with previously-described embodiments. All measuring appliances and connections used are, however, still easy to access.

An exemplary embodiment of a control cabinet for an exhaust gas measuring facility according to the present invention is illustrated in the drawings and is described below.

The control cabinet 10 for an exhaust gas measuring facility according to the present invention is composed of a first cabinet body 12 comprising a first side wall 14, an opposite second side wall 16, a bottom 18, a cover 20, as well as a rear wall 22. Inside this first cabinet body 12, a flame ionization detector analysis appliance 24 for determining hydrocarbons, a chemiluminescence detector analysis appliance 26 for determining nitrogen oxide quantities in the exhaust gas, as well as an infrared detector analysis appliance 28 for determining various active components, such as carbon monoxide, carbon dioxide or hydrocarbon compounds in the exhaust gas, are attached. These first measuring appliances 24, 26, 28 are connected to exhaust gas sample bags via pipes or directly to an exhaust gas measuring facility (not shown in the drawings).

For filling the exhaust gas sample bags or for creating the connection to the exhaust gas measuring facility (not shown in the drawings), openings are formed at the cover 20 or at the first side wall 14 and the second side wall 16 of the first cabinet body 12, in which openings either corresponding couplings for connecting the pipes from outside are formed or via which the pipes directly extend into the inside of the first cabinet body 12. The first measuring appliances 24, 26, 28 are similarly electrically connected to a power source. Electric cables are provided within the first cabinet body 12 for creating this connection and for creating an electrical connection to an operating unit 34 which is configured as a touchscreen monitor 35 and which is attached in an opening 36 of a door 38 which is rotatably attached to the first side wall 14 of the first cabinet body 12 via hinges 40 so that an open front side 42 of the first cabinet body 12 can be opened and closed. Both the exhaust gas measuring facility and the individual first measuring appliances 24, 26, 28 can be operated and their measured results can be read via this operating unit 34.

According to the present invention, an identically constructed second cabinet body 44 in its closed state is arranged behind the first cabinet body 12 and is connected to the first cabinet body 12 via articulated connections 46 in the form of hinges which are attached to the first side wall 14 of the first cabinet body 12 and to a first side wall 48 of the second cabinet body 44. This second cabinet body 44 accordingly does not include a door, but its front side 50 is opened by rotating the first cabinet body 12 about the articulated connections/hinges 46. Rollers 52 are attached to the bottom 18 of the first cabinet body 12 to provide this rotation and to fix the positioning of the second cabinet body 44. The second cabinet body 44 is closed via a corresponding closing device 54 which is arranged at the second side wall 16 of the first cabinet body 12 opposite to the first side wall 14 and at a second side wall 56 of the second cabinet body 44. After opening this closing device 54, the first cabinet body 12 can be rotated by 180° relative to the second cabinet body 44 so that the second measuring appliances 58, 60, 62 in the second cabinet body 44 are also fully accessible.

This second cabinet body 44 is not only constructed identically to the first cabinet body 12, i.e., it is configured with the two side walls 48, 56, a cover 64, a bottom 66, and a rear wall 68, it is also similarly equipped with a flame ionization detector analysis appliance 58, a chemiluminescence detector analysis appliance 60, as well as an infrared detector analysis appliance 62 as well as gas connecting pipes which are, however, fluidically connected to a parallel-connected second exhaust gas measuring facility. However, electric cables, via which the second measuring appliances 58, 60, 62 or the second exhaust gas measuring facility are controlled by the operating personnel, directly extend to the door 38 and the operating unit 34 arranged there at the first cabinet body 12.

The control cabinet according to the present invention is accordingly suitable for simultaneously controlling two exhaust gas measuring facilities by a single operator. The installation space required for this purpose is minimized by merely using one operating unit for two exhaust gas measuring facilities. All measuring appliances in the control cabinet are, however, still fully accessible. All units of the two measuring facilities are further spatially separated from each other and can, of course, be individually operated. The two cabinet bodies can be opened by simply being swung away from each other.

It should be appreciated that the scope of protection of the present invention is not limited to the described exemplary embodiment. The cabinets can in particular be equipped with other or additional measuring appliances, such as FTIR analysis appliances or quantum cascade lasers, CVS assemblies, analysis bags or particle collecting systems. The arrangement as well as the connections of the components to each other and to the respective exhaust gas measuring facility can also be changed. It is also possible to accommodate different measuring appliances for only one exhaust gas measuring facility in the two cabinet bodies and to provide, with the control cabinet according to the present invention, their accessibility at minimal space requirement. Reference should also be had to the appended claims.

What is claimed is:

1-9. (canceled)

10. A control cabinet for exhaust gas measuring facilities, the control cabinet comprising:
a first cabinet body configured to have first measuring appliances arranged therein for analyzing at least one sample gas;
a door configured to open and close an open front side of the first cabinet body, the door being rotatably attached to the first cabinet body; and
an operating unit arranged on the door;
a second cabinet body configured to have second measuring appliances arranged therein, the second cabinet body comprising a second front side that can be closed and opened via a guided relative movement of the first cabinet body in relation to the second cabinet body,
wherein,
the first cabinet body is movable in a guided manner relative to the second cabinet body.

11. The control cabinet as recited in claim 10, further comprising:
an articulated connection via which the second cabinet body is attached to the first cabinet body,
wherein,
the first cabinet body is arranged to be rotated about the articulated connection relative to the second cabinet body.

12. The control cabinet as recited in claim 10, wherein the second measuring appliances in the second cabinet body are electrically connected to the operating unit at the door.

13. The control cabinet as recited in claim 10, wherein,
the first measuring appliances in the first cabinet body are assigned to a first exhaust gas measuring facility, and
the second measuring appliances in the second cabinet body are assigned to a second exhaust gas measuring facility.

14. The control cabinet as recited in claim 10, wherein,
the first cabinet body comprises a bottom and rollers, and
the rollers are arranged at the bottom.

15. The control cabinet as recited in claim 10, wherein,
the first cabinet body comprises a rear wall, and
the rear wall rests against the second front side of the second cabinet body.

16. The control cabinet as recited in claim 10, wherein,
the first cabinet body and the second cabinet body are the same, and
the first measuring appliances and the second measuring appliances are the same.

17. The control cabinet as recited in claim 10, further comprising:
closing devices; and
a plurality of hinges which are configured to serve as an articulated connection,
wherein,
the first cabinet body comprises a first side wall and a second side wall which is arranged opposite to the first side wall,
the second cabinet body comprises a first side wall and a second side wall which is arranged opposite to the first side wall,
the plurality of hinges are arranged at the first side wall of the first cabinet body,
the first cabinet body is rotatably attached to the first side wall of the second cabinet body via the plurality of hinges arranged at the first side wall of the first cabinet body, and
one of the closing devices is arranged at each of the second side wall of the first cabinet body and at the second side wall of the second cabinet body so as to connect the second side wall of the first cabinet body to the second side wall of the second cabinet body.

18. The control cabinet as recited in claim 10, wherein,
the door comprises an opening, and
the operating unit comprises a monitor which is arranged in the opening of the door.
